(19) United States
(12) Patent Application Publication
(10) Pub. No.: US 2019/0125933 A1
(43) Pub. Date: May 2, 2019

(54) Electrostatic Charge-Induced Coating of Substrates with Biomolecules

(71) Applicant: Northeastern University, Boston, MA (US)

(72) Inventors: Garima BHARDWAJ, Allston, MA (US); Thomas J. WEBSTER, Barrington, RI (US)

(21) Appl. No.: 16/095,500

(22) PCT Filed: May 13, 2016
(86) PCT No.: PCT/US2016/032396

§ 371 Date: Oct. 22, 2018

(51) Int. Cl. A61L 29/08; A61L 29/06; A61L 29/16; B05D 1/18; B05D 3/14; C25D 13/04; C25D 13/14

(52) U.S. Cl. CPC A61L 29/08; A61L 29/06; A61L 29/085; A61L 29/16; B05D 1/18; A61L 2420/02; C25D 13/04; C25D 13/14; A61L 2300/404; A61L 2300/606; A61L 2400/18; B05D 3/14

(57) ABSTRACT

Methods, systems, and kits provide robust and biologically active coatings for implanted medical devices. The methods are based on electrostatic attraction between a conductive or non-conductive material surface on the medical device and a coating material including a charged biopolymer or pharmaceutical agent. Surface charge is induced or enhanced in the conductive or non-conductive material using a physical method. The methods are applicable to a wide variety of conductive or non-conductive substrate materials and coatings containing any of a wide variety of biological molecules and pharmaceutical agents.

### BACKGROUND

Coatings offer many improved features for implanted medical devices, including resistance to infection and the formation of biofilms, integration with tissue, promotion of healing, treatment or prevention of disease and harmful medical conditions, and the ability for long-term release of medications. Nevertheless, adding coatings to the surfaces of medical devices presents many challenges, such as lack of adhesion to certain materials, degradation or delamination of coating material, and the need to specially adapt coating chemistry to the substrate, to chemically modify the substrate, or to deposit multiple layers, often with considerable waste of expensive materials. Thus, there remains a need to develop simple, efficient, cost effective, and robust coatings and coating methods for medical devices.

### SUMMARY OF THE INVENTION

The invention provides methods, systems, and kits for coating medical devices and devices prepared thereby. The methods are based on electrostatic attraction between a conductive or non-conductive material surface on the substrate or medical device and a coating material including a charged biopolymer or pharmaceutical agent. Surface charge is induced or enhanced in the conductive or non-conductive substrate material using a physical method, and does not require chemical derivatization or pre-coating with a charged material. The methods are simple and applicable to a wide variety of conductive or non-conductive substrate materials and coatings containing any of a wide variety of biological molecules and pharmaceutical agents. The methods of the invention are unlike conventional coating methods that require chemical derivatization or pre-coating of conductive or non-conductive materials, or that require specifically tailored substrates or substrate treatments for each different type of coating material.

One aspect of the invention is a method of coating a substrate or medical device or a portion thereof. The method includes the steps of: (a) physically inducing or enhancing a surface charge on a conductive or non-conductive material at a surface of the medical device; and (b) depositing a coating material onto the surface, wherein the coating material has a charge which is opposite to the surface charge resulting from step (a). In some embodiments, the method further includes step (c) of depositing one or more additional layers of coating material onto the coating resulting from step (b). Preferred methods of physically inducing surface charge in step (a) include plasma treatment, laser treatment, ion bombardment, physical deposition, charging by friction, and charging by induction. In certain embodiments, excluded from step (a) are chemical functionalization or derivatization of the conductive or non-conductive material with a charged molecular species or coating with a charged molecular species, as well as applying an electrical potential to the medical device, so as to provide a surface charge. Preferred methods of depositing the coating material in step (b) are dip coating, electrophoretic deposition, spray coating, electrophoretic spray coating, spin coating, spin dipping, and sol-gel coating; more preferred are dip coating and electrophoretic deposition.

Another aspect of the invention is a system for coating a medical device according to the method described above. The system includes: (a) a medical device having an uncoated conductive or non-conductive material surface; (b) a device for physically inducing or enhancing a surface charge in the conductive or non-conductive material at the surface; and (c) a coating composition.

Yet another aspect of the invention is a coated medical device produced by the method or the system described above.

Still another aspect of the invention is a kit containing a medical device that includes a conductive or non-conductive material disposed at its surface. The kit further contains a charged coating material and instructions for inducing or enhancing a surface charge at the surface using a physical surface charge induction method and coating the charged conductive or non-conductive material surface of the device with the charged coating material. The charged coating material is a conductive or non-conductive material that has been electrostatically charged using a physical method that does not apply or leave any chemical residue on the surface and does not chemically derivatize the surface.

The invention can be further summarized with the following list of embodiments.

1. A method of coating a medical device or a portion thereof, the method comprising the steps of:

(a) physically inducing or enhancing a surface charge on a conductive or non-conductive material at a surface of the medical device; and

(b) depositing a coating material onto the surface, wherein the coating material has a charge which is opposite to the surface charge resulting from step (a).

2. The method of embodiment 1, wherein the surface material is non-conductive and comprises one or more materials selected from the group consisting of polyurethane, polyester, polyethylene, polyethylene terephthalate, polypropylene, polyamide, nylon, polycarbonate, polystyrene, poly(tetrafluoroethylene), poly(lactic-co-glycolic acid), poly(lactic-co-caprolactone), poly(lactide-co-glycolide), ceramic, glass, silicone, latex, rubber, and combinations thereof.
3. The method of embodiment 1, wherein the material is conductive and comprises one or more charged polymers selected from the group consisting of polymers bearing carboxylic, phosphonic, phosphoric, sulfuric, sulfonic, or amino groups, polyacids or polybases, polyamino acids, peptides, or proteins, polyamines, including poly(amino methacrylates), poly(dialkylaminoalkyl methacrylates), poly(dimethylaminoethyl methacrylate), poly(diethylaminoethyl methacrylate), polyvinylamines, polyvinylpyridines, quaternary polyvinylpyridines, poly(N-ethyl-4-vinylpyridine), poly(vinylbenzyltrimethylamines), polyallylamines, poly(allylamine hydrochloride, poly(diallyldialklylamines), poly(diallyldimethylammonium chloride), polyamidoamines, polyimines polyalkyleneimines, polyethyleneimines, polypropyleneimines, ethoxylated polyethyleneimines, hexadimethrene bromide, polycationic polysaccharides, chitosan, polysulfonates, polyvinylsulfonates, poly(styrenesulfonates), poly(sodium styrenesulfonate), sulfonated poly(tetrafluoroethylene), sulfonated styrene-ethylene/butylene-styrene triblock copolymers, sulfonated styrenic homopolymers and copolymers, polysulfates, polyvinylsulfates, sulfated and non-sulfated glycosaminoglycans, proteoglycans, heparin, heparin sulfate, chondroitin sulfate, keratan sulfate, dermatan sulfate, polyacrylates, methacrylates, methacrylic acid-ethyl acrylate copolymer, carboxymethylcellulose, carboxymethylamylose, polymers of mannuronic acid, galatcuronic acid and guluronic acid, alginic acid, hyaluronic acid, gelatin, carrageenan, polyphosphates, polyphosphonates, and polyvinylphosphonates.
4. The method of embodiment 1, wherein the material is conductive and comprises one or more metals selected from the group consisting of titanium, platinum, gold, tantalum, silver, copper, aluminum, palladium, stainless steel, nitinol, and alloys thereof.
5. The method of any of embodiments 1-4, wherein the coating material comprises a biomolecule selected from the group consisting of peptides, oligopeptides, polypeptides, proteins, glycoproteins, antibodies, enzymes, nucleic acids, nucleotides, oligonucleotides, polynucleotides, oligosaccharides, polysaccharides, lipids, glycosaminoglycans, proteoglycans, hormones, growth factors, cytokines, and combinations thereof.
6. The method of embodiment 5, wherein the biomolecule is antimicrobial, lubricious, or promotes attachment to cells or extracellular matrix.
7. The method of embodiment 6, wherein the biomolecule is a cationic peptide or cationic steroid.
8. The method of embodiment 7, wherein the biomolecule is a ceragenin.
9. The method of any of the preceding embodiments, wherein the coating comprises a pharmaceutical agent.
10. The method of embodiment 9, wherein the pharmaceutical agent is selected from the group consisting of antimicrobial agents, antibiotics, anticoagulants, anti-inflammatory agents, nitric oxide releasing agents, heparin, anti-tumor agents, and combinations thereof.
11. The method of any of the preceding embodiments, wherein step (a) comprises a method selected from the group consisting of plasma treatment, laser treatment, ion bombardment, physical deposition, charging by friction, charging by induction, and combinations thereof.
12. The method of any of the preceding embodiments, wherein step (a) does not include chemical functionalization or coating with a charged molecular species.
13. The method of any of the preceding embodiments, wherein step (a) does not include applying an electrical potential to the medical device.
14. The method of any of the preceding embodiments, wherein step (b) comprises a method selected from the group consisting of dip coating, electrophoretic deposition, spray coating, electrophoretic spray coating, spin coating, spin dipping, and sol-gel coating.
15. The method of embodiment 14, wherein step (b) comprises dip coating or electrophoretic deposition.
16. The method of any of the preceding embodiments, further comprising:

(c) depositing one or more additional layers of coating material onto the coating resulting from step (b).

17. The method of embodiment 16, wherein the one or more additional layers of coating material are the same as or different from the coating material applied in step (b).
18. A system for coating a medical device according to the method of any of the preceding embodiments, the system comprising:

(a) a medical device comprising an uncoated conductive or non-conductive surface;

(b) a device for physically inducing or enhancing a surface charge at said surface; and

(c) a coating composition.

19. The system of embodiment 18, wherein said surface comprises a surface material selected from the group consisting of polyurethane, polyester, polyethylene, polyethylene terephthalate, polypropylene, polyamide, nylon, polycarbonate, polystyrene, poly(tetrafluoroethylene), poly(lactic-co-glycolic acid), poly(lactic-co-caprolactone), poly(lactide-co-glycolide), ceramic, glass, silicone, latex, rubber, and combinations thereof.
20. The system of embodiment 18 or embodiment 19, wherein the coating composition comprises a biomolecule.
21. The system of any of embodiments 18-20, wherein the coating composition comprises a pharmaceutical agent.
22. The system of any of embodiments 18-21, wherein the device for inducing or enhancing an electrostatic charge is capable of performing a method selected from the group consisting of plasma treatment, laser treatment, ion bombardment, physical deposition, charging by friction, charging by induction, and combinations thereof.
23. A coated medical device produced by the method of any of embodiments 1-17 or the system of any of embodiments 16-20.
24. A kit comprising a medical device, the device comprising a conductive or non-conductive material disposed at a surface of the device, a charged coating material, and instructions for inducing or enhancing a surface charge at the surface using a physical surface charge induction method and coating the surface with the charged coating material.
25. The kit of embodiment 24, wherein the coating material comprises a biomolecule and/or a pharmaceutical agent.

### BRIEF DESCRIPTION OF THE DRAWINGS

FIG. 1 shows a schematic representation of a device in use for electrophoretic deposition of a coating on a non-conductive surface of a medical device.

FIGS. 2A-2C show scanning electron micrographs of the surface of a CA-13 coating deposited on a polyurethane endotracheal tube. The figures show increasing magnification from FIG. 2A (scale increments of 20 μm) to FIG. 2B (scale increments of 5 μm) to FIG. 2C (scale increments of 2 μm).

### DETAILED DESCRIPTION OF THE INVENTION

The present invention provides methods, systems, and kits for creating bioactive, uniform, and strongly-adherent coatings on medical devices to improve their performance. The invention utilizes induction or enhancement of electrostatic charge on the surface of a medical device, specifically on a surface of a conductive or non-conductive material of the medical device, by a physical method. The use of physical surface charge induction enables materials to be coated that are otherwise difficult or impossible to coat without complex and individualized chemical functionalization methods, and enables a variety of coating materials to be used with minimal or no individual adaptation. Uniform and thick coatings can be achieved on inside and outside surfaces, and without altering the inherent mechanical properties of the device. Biological activity of coating materials can be retained by employing gentle coating processes such as dip coating or electrophoretic deposition, without the need to chemically alter the coating material.

The invention is useful in commercial applications where, for example, it can prevent bacterial adhesion and proliferation, thereby reducing infection caused by implanted medical devices. The coating methods of the invention also can be used to promote bioactivity, reduce infection, and limit inflammation of medical devices such as hip implants and other joint replacements, cornea implants, pacemaker leads, and skin grafts. The methods of the invention provide ease of coating due to their simplicity and wide applicability, and are cost efficient. The invention also can be used to transform existing FDA-approved materials into materials that improve implant performance.

Any medical device that is implanted, in whole or in part, into a subject's body can be used in the invention. For example, suitable medical devices include, but are not limited to, catheters (e.g., vascular or balloon catheters), stents (e.g., biliary, coronary vascular, peripheral vascular, cerebral, esophageal, gastrointestinal, tracheal, urethral, or ureteral stents), cochlear implants, corneal implants, defibrillators, dental implants, endotracheal tubes, filters, guide wires, grafts, artificial hearts, heart valves, joint prostheses, leads, orthopedic screws, pins, and plates, ports, plugs, patches, pacemakers, pumps, surgical clips, staples, sutures, and meshes, tissue scaffolds, and vascular valves.

A subject can be any human or non-human animal subject, such as a mammalian subject, that is the recipient of a coated medical device provided by the invention.

In any method, system, device, or kit of the invention, the conductive or non-conductive material whose surface is coated can contain, for example, one or more charged or uncharged polymers or metals. For example, uncharged (i.e., non-conductive) polymers can include one or more materials selected from the group consisting of polyurethane, polyester, polyethylene, polyethylene terephthalate, polypropylene, polyamide, nylon, polycarbonate, polystyrene, poly(tetrafluoroethylene), poly(lactic-co-glycolic acid), poly(lactic-co-caprolactone), poly(lactide-co-glycolide), ceramic, glass, silicone, latex, rubber, and combinations thereof. In some non-conductive material embodiments, small amounts of conductive materials such as metals or conductive polymers also can be included in the non-conductive material; inclusions of such materials may aid in inducing or sustaining a surface charge prior to coating. Charged polymers can include, for example, polymers bearing carboxylic, phosphonic, phosphoric, sulfuric, sulfonic, or amino groups, polyacids or polybases, polyamino acids, peptides, or proteins, polyamines, including poly(amino methacrylates), poly(dialkylaminoalkyl methacrylates), poly(dimethylaminoethyl methacrylate), poly(diethylaminoethyl methacrylate), polyvinylamines, polyvinylpyridines, quaternary polyvinylpyridines, poly(N-ethyl-4-vinylpyridine), poly(vinylbenzyltrimethylamines), polyallylamines, poly(allylamine hydrochloride, poly(diallyldialklylamines), poly(diallyldimethylammonium chloride), polyamidoamines, polyimines polyalkyleneimines, polyethyleneimines, polypropyleneimines, ethoxylated polyethyleneimines, hexadimethrene bromide, polycationic polysaccharides, chitosan, polysulfonates, polyvinylsulfonates, poly(styrenesulfonates), poly(sodium styrenesulfonate), sulfonated poly(tetrafluoroethylene), sulfonated styrene-ethylene/butylene-styrene triblock copolymers, sulfonated styrenic homopolymers and copolymers, polysulfates, polyvinylsulfates, sulfated and non-sulfated glycosaminoglycans, proteoglycans, heparin, heparin sulfate, chondroitin sulfate, keratan sulfate, dermatan sulfate, polyacrylates, methacrylates, methacrylic acid-ethyl acrylate copolymer, carboxymethylcellulose, carboxymethylamylose, polymers of mannuronic acid, galatcuronic acid and guluronic acid, alginic acid, hyaluronic acid, gelatin, carrageenan, polyphosphates, polyphosphonates, and polyvinylphosphonates. Metal substrates for coating according to the invention include, for example, titanium, platinum, gold, tantalum, silver, copper, aluminum, palladium, stainless steel, nitinol, and alloys thereof. The conductive or non-conductive material can be biodegradable or non-biodegradable.

In any method, system, device, or kit of the invention, the coating material can contain, for example, a biomolecule selected from the group consisting of peptides, oligopeptides, polypeptides, proteins, glycoproteins, antibodies, enzymes, nucleic acids, nucleotides, oligonucleotides, polynucleotides, oligosaccharides, polysaccharides, lipids, glycosaminoglycans, proteoglycans, hormones, growth factors, and cytokines.

In any method, system, device, or kit of the invention, the coating material can contain, for example, a pharmaceutical agent selected from the group consisting of antimicrobial agents, antibiotics, anticoagulants, anti-inflammatory agents, nitric oxide releasing agents, heparin, anti-tumor agents, and combinations thereof.

The coating material can further include one or more components to aid in adhesion of the coating to the medical device or to cells or extracellular matrix components within the body of the subject, stability of the biomolecule, release of the biomolecule, function of the biomolecule, stability or degradation of the coating, lubricity, opacity to X-rays, and the like. Further, the coating material can include pharmaceutical formulation components, such as excipients, diluents, carriers, buffers, delayed release agents, dispersants, surfactants, emulsifiers, or antioxidants.

Many of the materials used in the invention are polymers, either organic or inorganic. Such polymers can be used either as components of a medical device, or forming an entire medical device, as a substrate or surface material for a medical device, as a structural element exposed on the surface of a medical device, or as components of a coating composition, or as pharmaceutical formulation ingredients. Polymers for coating according to the invention can be linear, branched, cyclic, or can have a comb or dendritic configuration. Polymers for coating according to the invention also can be homopolymers or copolymers, such as block copolymers. Polymers can be uncharged or charged. Non-polymeric components or materials found in a medical device for coating according to the invention, such as metals, also can be used in the present invention as a substrate to which a surface charge is applied by a physical method, and which is then coated with a biopolymer.

In one embodiment of a method of coating a medical device according to the present invention, the medical device includes at least a portion or component having a non-conductive material at its surface. Unlike metals or other conductive materials, it is difficult or impossible to establish a surface potential on a non-conductive material by simply using the material as an electrode in an electrophoretic or dip-coating deposition process, i.e., by applying an electrical potential between the material and a solution containing charged molecules of a coating material. In the present invention, other physical methods are used to impart an electrostatic charge, i.e., surface potential or surface charge, on an exposed surface of a non-conductive material of a medical device.

Preferred methods of physically inducing surface charge in a conductive or non-conductive material include plasma treatment, laser treatment, ion bombardment, physical deposition methods, charging by friction, and charging by induction. These are well known methods that are capable of inducing an electrical charge at the surface of a material. Furthermore, it is well understood how to practice such methods so as to induce a net positive or a net negative charge.

Exposure of conductive or non-conductive materials to a plasma, such as an oxygen or argon plasma, can induce a surface charge or enhance a pre-existing surface charge. Plasma treatment such as plasma etching involves exposing the material to atoms of a plasma typically formed as a high-speed stream of atoms of a gas or mixture of gases directed onto the material. The plasma source can deliver charged atoms or radicals onto the surface of the material. The plasma atoms can embed in the material and/or form reactive moieties in the material, which alter the surface charge, and optionally also the polarity of the surface, rendering hydrophobic surfaces more hydrophilic and more wettable. Physical deposition methods, such as sputtering, also can be used to introduce atoms onto the surface of a conductive or non-conductive material so as to increase the surface charge, as can bombardment with an ion beam or exposure to a laser.

Charging by friction and charging by induction are also suitable methods for surface charge induction. Charging by friction uses the triboelectric effect, or contact electrification. In charging by friction, a charge-inducing material having greater or lesser electron affinity in comparison to the conductive or non-conductive material is rubbed across the surface of the conductive or non-conductive material. If the charge inducing material has greater electron affinity, then in will render the surface of the conductive or non-conductive material positive. If the charge inducing material has lesser electron affinity, then it will render the surface of the conductive or non-conductive material negative. The relative electron affinities of different materials is known and can be found in the literature as triboelectric series, or can be measured, to aid in selecting a suitable material for charging any given conductive or non-conductive material either positively or negatively. In charging by induction, friction is not applied to the conductive or non-conductive surface, but instead an already charged object is brought into proximity with the conductive or non-conductive material, but without contacting it, and either the conductive or non-conductive material is grounded or another object in contact with the conductive or non-conductive material is removed, resulting in charging of the conductive or non-conductive material.

While the invention utilizes primarily physical mechanisms of inducing surface charge, it is understood that a consequence of such physical mechanisms can be that a variety of random chemical reactions are induced in the material. This is distinguished, however, from purely chemical mechanisms in which a specific desired chemical reaction is induced by providing specific reactants and causing a specific chemical reaction to occur. Such purely chemical mechanisms are generally excluded from the present invention. In certain embodiments, however, purely chemical mechanisms may be used in conjunction with primarily physical mechanisms.

The extent of surface charge induction or enhancement may vary depending on the method used. Any amount of surface charge induced, or increased over pre-existing levels, is considered within the invention provided that it results in a measurable increase in coating thickness, completeness of coverage, robustness, durability in use, or biological or pharmacological activity compared to a coating that results without the surface charge induction or enhancement. Surface charge can be measured as an electrical potential difference between an outer surface of a conductive or non-conductive material of a medical device and the interior of the conductive or non-conductive material. The surface charge can be affected by the ionic environment of the material; it is therefore preferable to determine the surface charge under conditions used to deposit the coating material, i.e., in a solution used for dip-coating or electrophoretic deposition. Surface charge can also be represented by the surface charge density, which is the amount of electrical charge per unit of surface area.

Once the surface charge of the conductive or non-conductive material surface of the medical device has been induced or enhanced, any method can be used to deposit the coating material on that surface, or region of a surface. However, the employed method should preserve the desired biological activity or pharmacological activity provided by the coating. Therefore, preferred coating methods include dip coating, electrophoretic deposition, spray coating, electrophoretic spray coating, spin coating, spin dipping, and sol-gel coating, because these methods can apply the coating material in the form of a solution containing and preserving the activity of the biological or pharmaceutical agent.

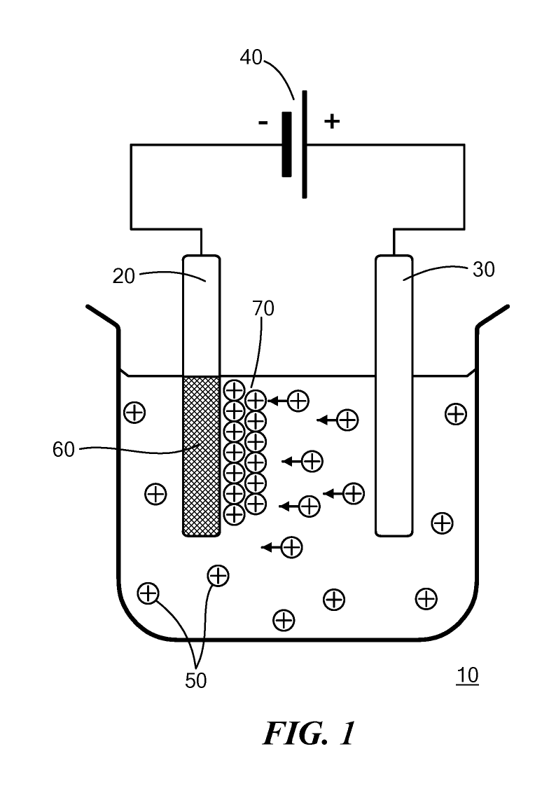

One example of an apparatus for electrophoretic deposition is shown in FIG. 1. Apparatus 10 includes cathode 20 and anode 30 which are immersed in an electrolyte solution containing molecules of coating material 50. The electrodes are set at a potential controlled by voltage source 40, which can be a DC or AC power supply or a battery. In this embodiment, tube 60, such as an endotracheal tube, surrounds the cathode with a loose fit, so that electrolyte solution contacts both inner and outer surfaces of the tube, and a coating can be deposited on all surfaces of the device. Also in this embodiment, tube 60 has been provided with a negative surface charge, so that cationic coating molecules are attracted to it to form coating 70.

### EXAMPLES

### Example 1. Application by Dip Coating of an Antimicrobial Coating After Surface Charge Induction

Polyurethane endotracheal tubes were coated with a compound known to have bactericidal activity (CSA-13). A positive electrostatic charge was first induced on both the inside and outside surfaces of the tubes by rubbing them with wool for 5 minutes each. Then, the tubes were coated with CSA-13 by dip coating. For dip coating, the charged tubes were dipped in a CSA-13 solution three times, each time for one minute. The result was a thick and uniform coating, if the surface of the tube previously had been charged, and no coating if the surface of the tube had not been charged.

### Example 2. Application by Electrophoretic Deposition of an Antimicrobial Coating After Surface Charge Induction

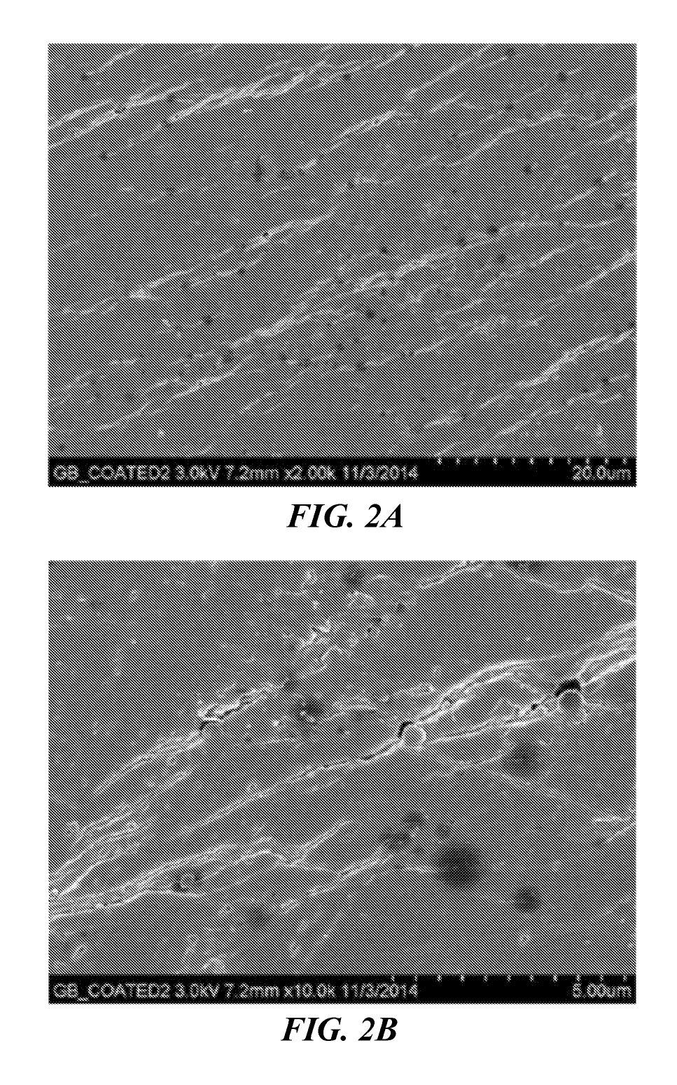

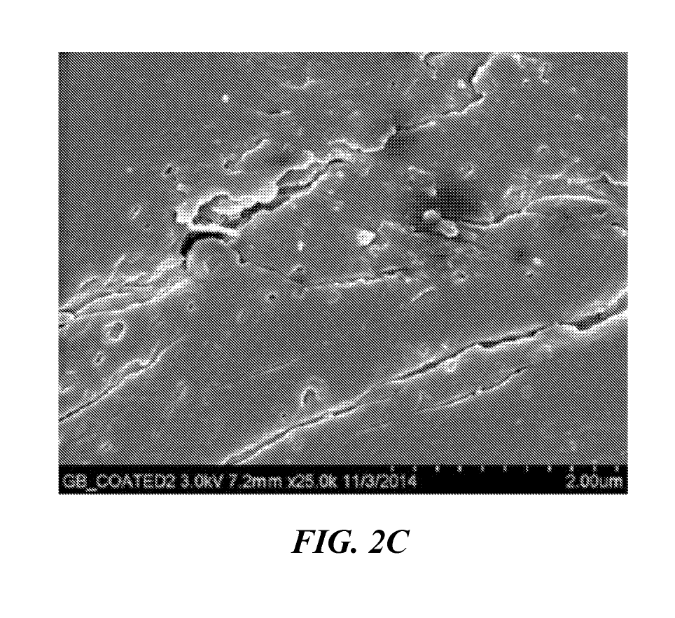

Polyurethane endotracheal tubes were coated with CSA-13 using electrophoretic deposition. A positive electrostatic charge was first induced on both the inside and outside surfaces of the tubes by rubbing them with wool for 5 minutes each, which was followed by electrophoretic deposition using an apparatus similar to that depicted in FIG. 1. Titanium foil purchased from Alfa Aesar (catalog number 10385) was used as both the counter and working electrodes, and the CSA-13 solution was used as the electrolyte. The polyurethane tube was placed around the titanium foil that functioned as the negative electrode, and this allowed the compound to coat the tube surface both inside and out. The voltage used was 20 V for a time period of 10 minutes. Tubes whose surface had previously been charged became coated with CSA-13, but no coating was observed if no charge was induced prior to the coating procedure. The tubes were removed from the apparatus and then allowed to dry at room temperature. The coated tubes were visualized using scanning electron microscopy (SEM) to reveal the morphology of the surface coating. A uniform coating was observed (see FIGS. 2A-2C) having a smooth surface, with occasional cracks.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

1. A method of coating a medical device or a portion thereof, the method comprising the steps of:
(a) physically inducing or enhancing a surface charge on a conductive or non-conductive material at a surface of the medical device; and
(b) depositing a coating material onto the surface, wherein the coating material has a charge which is opposite to the surface charge resulting from step (a).

2. The method of claim 1, wherein the surface material is non-conductive and comprises one or more materials selected from the group consisting of polyurethane, polyester, polyethylene, polyethylene terephthalate, polypropylene, polyamide, nylon, polycarbonate, polystyrene, poly(tetrafluoroethylene), poly(lactic-co-glycolic acid), poly(lactic-co-caprolactone), poly(lactide-co-glycolide), ceramic, glass, silicone, latex, rubber, and combinations thereof.

3. The method of claim 1, wherein the material is conductive and comprises one or more charged polymers selected from the group consisting of polymers bearing carboxylic, phosphonic, phosphoric, sulfuric, sulfonic, or amino groups, polyacids or polybases, polyamino acids, peptides, or proteins, polyamines, including poly(amino methacrylates), poly(dialkylaminoalkyl methacrylates), poly(dimethylaminoethyl methacrylate), poly(diethylaminoethyl methacrylate), polyvinylamines, polyvinylpyridines, quaternary polyvinylpyridines, poly(N-ethyl-4-vinylpyridine), poly(vinylbenzyltrimethylamines), polyallylamines, poly(allylamine hydrochloride, poly(diallyldialklylamines), poly(diallyldimethylammonium chloride), polyamidoamines, polyimines polyalkyleneimines, polyethyleneimines, polypropyleneimines, ethoxylated polyethyleneimines, hexadimethrene bromide, polycationic polysaccharides, chitosan, polysulfonates, polyvinylsulfonates, poly(styrenesulfonates), poly(sodium styrenesulfonate), sulfonated poly(tetrafluoroethylene), sulfonated styrene-ethylene/butylene-styrene triblock copolymers, sulfonated styrenic homopolymers and copolymers, polysulfates, polyvinylsulfates, sulfated and non-sulfated glycosaminoglycans, proteoglycans, heparin, heparin sulfate, chondroitin sulfate, keratan sulfate, dermatan sulfate, polyacrylates, methacrylates, methacrylic acid-ethyl acrylate copolymer, carboxymethylcellulose, carboxymethylamylose, polymers of mannuronic acid, galatcuronic acid and guluronic acid, alginic acid, hyaluronic acid, gelatin, carrageenan, polyphosphates, polyphosphonates, and polyvinylphosphonates.

4. The method of claim 1, wherein the material is conductive and comprises one or more metals selected from the group consisting of titanium, platinum, gold, tantalum, silver, copper, aluminum, palladium, stainless steel, nitinol, and alloys thereof.

5. The method of claim 1, wherein the coating material comprises a biomolecule selected from the group consisting of peptides, oligopeptides, polypeptides, proteins, glycoproteins, antibodies, enzymes, nucleic acids, nucleotides, oligonucleotides, polynucleotides, oligosaccharides, polysaccharides, lipids, glycosaminoglycans, proteoglycans, hormones, growth factors, cytokines, and combinations thereof.

6. The method of claim 5, wherein the biomolecule is antimicrobial, lubricious, or promotes attachment to cells or extracellular matrix.

7. The method of claim 6, wherein the biomolecule is a cationic peptide or cationic steroid.

8. The method of claim 7, wherein the biomolecule is a ceragenin.

9. The method of claim 1, wherein the coating comprises a pharmaceutical agent.

10. The method of claim 9, wherein the pharmaceutical agent is selected from the group consisting of antimicrobial agents, antibiotics, anticoagulants, anti-inflammatory agents, nitric oxide releasing agents, heparin, anti-tumor agents, and combinations thereof.

11. The method of claim 1, wherein step (a) comprises a method selected from the group consisting of plasma treatment, laser treatment, ion bombardment, physical deposition, charging by friction, charging by induction, and combinations thereof.

12. The method of claim 1, wherein step (a) does not include chemical functionalization or coating with a charged molecular species.

13. The method of claim 1, wherein step (a) does not include applying an electrical potential to the medical device.

14. The method of claim 1, wherein step (b) comprises a method selected from the group consisting of dip coating, electrophoretic deposition, spray coating, electrophoretic spray coating, spin coating, spin dipping, and sol-gel coating.

15. The method of claim 14, wherein step (b) comprises dip coating or electrophoretic deposition.

16. The method of claim 1, further comprising:
(c) depositing one or more additional layers of coating material onto the coating resulting from step (b).

17. The method of claim 16, wherein the one or more additional layers of coating material are the same as or different from the coating material applied in step (b).

18. A system for coating a medical device according to the method of claim 1, the system comprising:
(a) a medical device comprising an uncoated conductive or non-conductive surface;
(b) a device for physically inducing or enhancing a surface charge at said surface; and
(c) a coating composition.

19. The system of claim 18, wherein said surface comprises a surface material selected from the group consisting of polyurethane, polyester, polyethylene, polyethylene terephthalate, polypropylene, polyamide, nylon, polycarbonate, polystyrene, poly(tetrafluoroethylene), poly(lactic-co-glycolic acid), poly(lactic-co-caprolactone), poly(lactide-co-glycolide), ceramic, glass, silicone, latex, rubber, and combinations thereof.

20. The system of claim 18, wherein the coating composition comprises a biomolecule.

21. The system of claim 18, wherein the coating composition comprises a pharmaceutical agent.

22. The system of claim 18, wherein the device for inducing or enhancing an electrostatic charge is capable of performing a method selected from the group consisting of plasma treatment, laser treatment, ion bombardment, physical deposition, charging by friction, charging by induction, and combinations thereof.

23. A coated medical device produced by the method of claim 1.

24. A kit comprising a medical device, the device comprising a conductive or non-conductive material disposed at a surface of the device, a charged coating material, and instructions for inducing or enhancing a surface charge at the surface using a physical surface charge induction method and coating the surface with the charged coating material.

25. The kit of claim 24, wherein the coating material comprises a biomolecule and/or a pharmaceutical agent.
